(19) United States
(12) Patent Application Publication
(10) Pub. No.: US 2010/0234755 A1
(43) Pub. Date: Sep. 16, 2010

(54) Electrode holder for use on hairy animals such as horses, camels, and the like

(76) Inventors: Neal S. Latman, Amarillo, TX (US); Joseph E. Batson, JR., Amarillo, TX (US); Alan Nicholson, Canyon, TX (US); Ladislado Garza, Amarillo, TX (US); Mike Floyd, Amarillo, TX (US)

Correspondence Address: Harold H. Flanders, 904 Sweetwater Cove, Round Rock, TX, 78681, US

(21) Appl. No.: 12/381,353

(22) Filed: Mar. 11, 2009

(51) Int. Cl. A61B 5/05

(52) U.S. Cl. 600/547

(57) ABSTRACT

An electrode holder comprising a pair of electrodes connected by a flexible, insulating rod which controls the position of the electrodes with respect to each other and the topographic surfaces of the body or body part of the animal.

### RIGHTS TO INVENTION UNDER FEDERAL SPONSORED RESEARCH OR DEVELOPMENT

None

### CROSS REFERENCE TO RELATED APPLICATIONS

The following applications co-owned by the same inventive entity were filed of even date herewith: CCC 0806 B, 0807EQ, and 0808B.

### REFERENCE TO MICROFICHE APPENDIX

None

### BACKGROUND OF THE INVENTION

1. Field of the Invention

The present invention relates generally to a process applicable to electrodes and systems for holding electrodes and more particularly to a new and improved method for the use of such electrodes and holders in connection with hairy animals such as horses, camels, and the like in Bioelectrical Impedance Analysis (BIA), and for such other structures, apparatus, processes, systems, and methods as may be herein disclosed.

The present invention is and may be used in connection with measurements requiring the use of electrodes as described herein and in general.

As a specific example reference is made to Bioelectrical Impedance Analysis (BIA). The initials or acronym BIA may refer to one or more of several slightly different terms all of which may be considered to be equivalent as discussed below and used herein. The meaning is almost always clear from the context of use and for the purposes of this application these terms may in general be used interchangeably. The term Bioelectrical Impedance Analysis is generally preferred and used herein as it conveys the most information in its terms. Other generally equivalent terms which may be used include Bioimpedance Analysis or BioImpedance Analysis, Biological Impedance Analysis, Biological Impedance Interface, Electrical Bio-impedance, Electrical Impedance Analysis, and similar terms and combination of terms.

2. Relevant State of the Art and Description of Related Prior Art

As has been noted above, the present invention relates to electrodes in general and especially to measurements as may be required on hairy animals such as horses, camels, and the like.

Horses and camels are important animals having economic, agricultural, sports and entertainment significance. Various measurements may be made or desired relating to their training, working, and breeding. Similar concerns and measurements may be made on other hairy animals such as camels.

Among the measurements of particular interest are those known as bioelectrical impedance analysis, although as has been noted the present invention is not limited to such specific measurements or techniques.

Bioelectrical Impedance Analysis may be used to measure and analyze a wide range of ionic and charge transfer processes in bio-materials and biological systems in general.

As a matter of general background to the present invention, it may be helpful to note the following terms:

Electricity is the movement of electrons. Electrons have a negative charge. Free electrons will flow or move towards a positive charge or down an electrical gradient towards a less negative charge.

Amperes (Amps) is the number of free electrons flowing or moving per unit time. Sometimes this flow of electrons is referred to as “Intensity” or “I”. Sometimes this flow is referred to as “electrical current”. In many situations, it may be best to think about amperes as the amount of or volume of electrons that are moving per unit of time. One ampere=6.25×10¹⁸ electrons per second.

Conductors: The flow of electrons moves along a material or substance called a “conductor”. Some substances offer more or less resistance to the flow of electrons than others. Those that offer little resistance to the flow of electrons are considered “good conductors” A good conductor is a material that has electrons that are less tightly bound and therefore, more free to move. In a bad conductor, the electrons are more tightly bound and less free to move. A really bad conductor is called an “insulator”.

Volts represent the potential difference in charges between two points in or along a conductor. That means that there is an electrical gradient between the two points. In other words, there are more negative charges (electrons) at one point than at the other. The more positively charged point would exert an attractive force or pull on the electrons toward it. The attractive force is called an “electromotive force”.

The relationship between amperes, volts and resistance to flow of electrons may be expressed by Ohm's law: Volts=Amperes×Resistance in Ohms. All conductors offer some resistance to the flow of electrons.

A “capacitor in an electric circuit is a non-conductor (insulator, sometimes called a “dielectric”) that is sandwiched between two conductors. As the electrons flow down the conductor, it comes to the capacitor. Because the capacitor is a non-conductor, the electrons begin to pile-up on one side of it. As more negatively charged electrons accumulate, the potential electrical difference between the negative side of the capacitor and the relatively positively charged side increases. Like charges repel each other. So, as the negatively charged electrons accumulate on one side of the capacitor, the increasing negative charge on that side of the capacitor repels the negatively charged electrons on the other side of the capacitor. That results in one side of the capacitor with more electrons next to the capacitor than the other side. When the potential difference in negative electrons between the two sides is sufficiently great, the electrons on the relatively less negative side of the capacitor begin to move away from the capacitor and down the conductor. We can view a capacitor as a non-conductor that results in an increase in voltage.

Sometimes, capacitance is thought of as the amount of electrons necessary to raise the potential by a specific amount. At other times, capacitance may be thought of as the amount of electrons that can be “stored” on a surface (i.e., the negative side of the capacitor), before the electrical current moves on. Capacitance is measured in “Farads”

A biological cell membrane is composed of a biomolecular layer of phospholipids. Lipids are poor electrical conductors. They are so poor as to be viewed as non-conductors. When an electrical current flows through the fluids in the body (a relatively good conductor) and comes to a cell membrane such as a red blood cell, the cell membrane acts as a capacitor, the capacitance of which can be measured.

Bioelectrical Impedance Analysis (BIA) measures the impedance or opposition to the flow of electrical current through body fluids. Impedance is low in lean tissue where intracellular fluid and electrolytes are primarily contained, but high in fat tissue. Impedance is generally proportional to body water volume. In practice a small constant current, typically 800 uA at a fixed frequency, for example 50 kHz, is passed between electrodes spanning the body parts in question and the voltage drop between electrodes provides a measure of impedance.

The impedance of a biological tissue comprises two components, the resistance and the reactance. The conductive characteristics of body fluids provide the resistive component, whereas the cell membranes, acting as imperfect capacitors, contribute a frequency-dependent reactive component.

Impedance measurements made over a range of low to high (1 MHz) frequencies, allow the development of predictive equations. For example equations may relate impedance measures at low frequencies to extracellular fluid volumes and at high frequencies to total body fluid volume. This approach is known a multi-frequency bioelectrical impedance analysis (MFBIA).

The BIA measurements in general involve the measurement of:
a.) resistance in ohms {“R”}
b.) reactance in ohms {“Xc”} [basically defined as the opposition to transmission of electrical energy through a capacitor.]
c.) impedance in ohms {“Z”} [basically defined as Z=√[R²+(Xc)²] (i.e. the square root of [R squared+Xc squared]).

The above paraphrased from tutorial papers of Dr. Neal Latman and from pp. 29-32 Horowitz & Hill, The Art of Electronics (2d Ed.) Cambridge University Press, Cambridge, Mass., 1989.

The background of the present invention is more specifically provided by Forro, Mariam, Scott Cieslar, Gayle L. Ecker, Angela Walzak, Joy Hahn, and Michael I Lindenger, “Total body water and ECFV measured using bioelectrical impedance analysis and indicator dilution in horses”: J. Appl Physiol 89: 663*671,2000. which to some extent appears to teach away from the present invention, see sections on the linear regression analysis.

Also see, Fielding, C. Langdon, Gary Magdesean, Denise A. Elliott, Larry D. Cowgell, and Gary P. Carlson; “Use of multifrequency bioelectrical impedance analysis for estimation of total body water and extracellular and intracellular fluid volumes in horses”, AJVR, Vol 65, No. 3, 320, 326 March 2004.

See also U.S. Pat. No. 6,850,798 which measures animal body fat via the hooves and foot pads; U.S. Pat. Nos. 6,308,096 and 6,321,112 at their FIG. 25 and 2001/0007055 which purports to measure fatigue, see FIG. 12.

U.S. Pat. No. 6,360,124 is handheld and U.S. Pat. No. 6,400,983 which employs hand electrodes.

U.S. Pat. No. 6,477,409 measures metabolism and U.S. Pat. No. 6,487,445 utilizes calipers.

U.S. Pat. Nos. 6,490,481; 6,509,748; and 2003/0216665 employ multiple electrodes with other body data while U.S. Pat. Nos. 6,516,221 and 6,725,089 feature graphic displays.

U.S. Pat. No. 6,567,692 utilizes multiple sites, U.S. Pat. No. 6,621,013 selects body information to be evaluated.

2003/0176808 allows for multiple fat layers.

2004/0019292 permits use in identification.

2004/0171963 and 2005/0059902 focus on body composition and 2004/0236245 on muscle mass.

2005/0124909 is directed to the measurement of body fat in animals.

U.S. Pat. No. 6,978.170 focuses on electrode positioning.

2006/0094979 and 2006/0111645 utilize multiple pairs of electrode systems.

Other references of interest include:

U.S. Pat. Nos. 3,602,215; 3,851,641; 3,871,359; 3,971,365; 4,008,712; 4,116,231; 4,336,873; 4,377,170; 4,423,792; 4,144,763; 4,557,271; 4,557,271; 4,493,362; 4,578,635; 4,557,271; 4,773,492; 4,831,242; 4,831,527; 4,844,187; 4,947,862; 4,805,621; 4,895,163; 4,911,175; 4,919,145; 4,947,862; 5,063,937; 5,086,781; 5,203,344; 5,579,782; 6,088,615; 6,208,890; 5,722,396; 5,819,741; 6,004,312; 6,188,925; 6,280,396; 6,308,096; 6,354,996; 6,370,425; 6,393,317; 6,400,983; 4,949,727; 5,052,405; 5,105,825; 5,372,141; 5,458,117; 5,720,296; 5,746,214; 5,817,031; 5,840,042; 6,151,523; 6,198,964; 6,256,532; 6,265,882; 5,483,970; 5,335,667; 5,415,176; 5,435,115; 5,449,000; 5,595,189; 5,611,351; 5,615,689; 5,749,369; 5,335,667; 5,817,031; 6,088,615; 6,292,690; 2002/0026173; U.S. Pat. Nos. 6,370,425; 6,393,317; 2002/0151815; U.S. Pat. No. 6,473,643′2002/0151311; U.S. Pat. No. 6,631,292; 2004/0002662; U.S. Pat. Nos. 5,088,489; 5,335,667; 5,718,850; 5,720,296; 5,729,905; 6,038,465; 6,088,615; 6,321,112; 6,398,740; 6,440,068; 6,327,495; 5,371,469; 5,483,970; 5,503,157; 5,865,763; 6,011,992; 6,339,722; 6,442,422; 6,450,955; 6,490,481; 6,487,445; 6,516,221; 6,526,315; 6,567,692; 5,579,782; 5,819,741; 6,004,312; 6,168,563; 6,280,396; 6,308,096; 6,685,654; 2004/0077968; U.S. Pat. No. 6,752,760; 2004/0260196; U.S. Pat. No. 6,865,415; 2005/0059903/; 2005/0080352; U.S. Pat. No. 6,889,076; 2005/0101875; 2005/0171451; 2005/0177060; 2005/0177062; 2005/0192488; 2005/0209528; 2006/0025701; and 2006/0094978.

While a number of Bioelectrical Impedance Analysis (BIA) systems are shown and taught by the above art they, in general, fail to recognize the importance of the electrode system and its critical significance.

### BRIEF SUMMARY OF THE INVENTION

The present invention relates to electrodes and electrode holders for use in connection with measurements on hairy animals such as horses, camels, and the like.

The present invention may be used for information relative to racing, training, working and breeding. Whether the horse is involved in racing, rodeos, jumping or other competitions; ranch and farm work, reproductive services; or as a pet, the hydration status of the horse is important to the horse, to the horse's owner, and to the horse's trainer and veterinarian.

A change of hydration of as little as one (1%) percent can affect the performance of the horse. Determination of the optimal hydration status for each horse is important, if the horse is to be maintained at the optimal level of hydration to ensure optimal performance. Dehydration of five (5%) percent or more is clinically significant. When dehydrated, blood pressure, heart rate, muscle function and the ability to perspire and maintain the desired body temperature is affected. Severe dehydration is life threatening.

The present invention while designed primarily for use with horses and camels can be used with other hairy animals including but not limited to cattle, hogs and dogs.

The present invention can be utilized to measure, or to be a part of a system to measure or evaluate such parameters as total body water, extra and intra cellular fluid, plasma, lean muscle mass, fat, the extent of marbling, phase angles, and general, overall health. The present invention can provide both preventative analysis and the detection of contamination and out of balance states of hydration

### Objects

Pursuant to the foregoing, it may be regarded as an object of the present invention to overcome the deficiencies of and provide for improvements in the state of the prior art as described above and as may be inherent in the same or as may be known to those skilled in the art.

It is a further object of the present invention to provide a process and any necessary apparatus for carrying out the same and of the forgoing character and in accordance with the above objects which may be readily carried out with and within the process and with comparatively simple equipment and with relatively simple engineering requirements.

Still further objects may be recognized and become apparent upon consideration of the following specification, taken as a whole, in conjunction with the appended drawings and claims, wherein by way of illustration and example, an embodiment of the present invention is disclosed related to hairy animals such as horses and camels, and the bioelectrical impedance analysis of the same.

As used herein, any reference to an object of the present invention should be understood to refer to solutions and advantages of the present invention which flow from its conception and reduction to practice and not to any a priori or prior art conception.

The above and other objects of the present invention are realized and the limitations of the prior art are overcome by providing a new and improved apparatus, methods and processes applicable to measurements to be made on hairy animals such as horses and camels.

### Technical Problems to be Solved

The need for an electrode and electrode holding system to provide accurate, reliable and repeatable measurements has long existed and been an unfulfilled need prior to the invention of the present apparatus and process.

In particular, the uneven topographic surfaces presented by certain animals such as horses and the like which may have abundant hair coats over uneven muscles and bone structures have long presented a problem of obtaining accurate and reproducible measurements in various electrical systems including those directed to bioelectrical impedance analysis.

### BRIEF DESCRIPTION OF THE DRAWINGS AND THEIR SEVERAL VIEWS

The above mentioned and other objects and advantages of the present invention and a better understanding of the principles and details of the present invention will be evident from description taken in conjunction with the appended drawings.

The drawings constitute a part of this specification and include exemplary embodiments of the present invention, which may be embodied in various forms. It is to be understood that in some instances various aspects of the invention may be shown as exaggerated, reduced, or enlarged or otherwise distorted to facilitate an understanding of the present invention.

In the drawings appended hereto:

FIG. 1 is a top view of the present invention.

FIG. 2 is a side view of the present invention.

FIG. 3 is a front view of the present invention.

In the accompanying drawings, like elements are given the same or analogous references when convenient or helpful for clarity. The same or analogous reference to these elements will be made in the body of the specification, but other names and terminology may also be employed to further explain the present invention.

### GENERAL DESCRIPTION OF THE INVENTION, DETAILED DESCRIPTION OF PREFERRED EMBODIMENTS OF AND BEST MODE FOR CARRYING OUT THE PRESENT INVENTION

The present invention relates to electrodes and electrode holders for use on animals in Bioelectrical Impedance Analysis (BIA) and other electrical measurements. The electrical measurements may be directed to any of a number of areas of electrical activities either of endogenous or exogenous origin. The preferred electrodes are for use on hairy animals such as a horses, camels, and/or the like. The electrodes are of the surface electrode type which do not, and are not intended to, penetrate the skin. The electrodes systems are intended to overcome problems in the prior art as noted above. They will provide a defined distance between the surface contact points of the electrodes. They will also be flexible enough to adjust to uneven surfaces, contours and topology of the animal. The electrodes and their holders are designed to provide for movement of the electrodes to overcome surface topography and to provide the other needs in providing accurate and reproducible measurements.

The present invention may be used and/or adapted for use in any Bioelectrical Impedance System. One preferred system is that known as a tetra-polar system which usually will employ two (2) electrodes at each end of a hairy animal.

The connecting rod is an insulating flexible rod on the order of 5-10 cm in length. The rod may be a relatively large diameter wire or rod of copper, carbon, silver, gold, platinum or other suitable flexible material. The rod may be a conductor suitably insulated or a non-conductive, electrically insulating material.

The electrodes may be coated with a conducting cream or gel to improve contact with the animal skin. The electrode holder may optionally include a compartment for containing and dispensing such cream or gel.

The electrodes may have attachment clips of various known designs.

The electrodes may be covered with elastomeric conformable or malleable conductive material having physical properties similar to some known silicone products. Some of the conformable materials may be assisted in their properties by the heat of the animals skin.

The system is not limited to hard wired transmission of its power voltage or signals.

The flexible rods may consist of insulating plastic tubing surrounding a concentric internal carbon fiber or other suitably flexible rod.

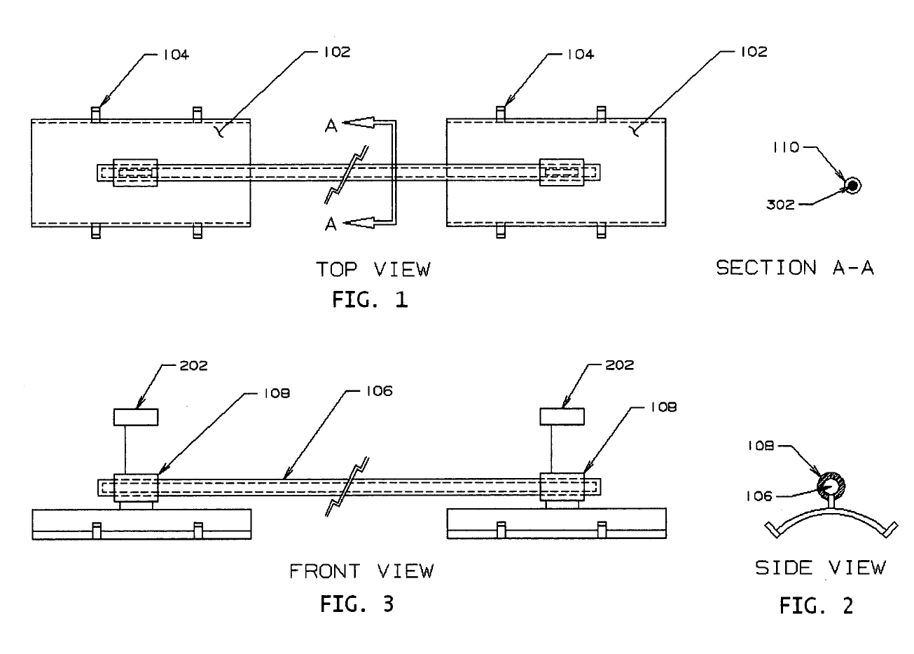

As shown in FIG. 1 electrodes 102 are positioned and attached by flexible rod 106 which is an insulating rod or a coated rod of insulating materials around a conductive or non-conductive rod or core 302 (See FIG. 3) of carbon or other suitable material.

The electrodes 102 have detents or hooks 104 to allow an elastomeric material (such as an elastic bandage) to be attached to electrodes 102 and wrapped around a limb or other body part of the animal. The rod 106 is attached to the electrode 102 by clips 108 which engage the rod 302 and are held in place by the insulating material 110 on rod 106. The clip 108 is in electrical contact with the electrode 102.

The electrode 102 is curved to conform to the animal leg or other body part.

Other electrical clips and attachments may be clipped to clip or strap 108 which is attached to electrode 102. The electrode 102 may be hardwired to the appropriate electrical input for BIA or other measurements. Additionally the electrode holder can provide a base on which can be mounted an electro-magnetic energy power supply (such as electricity) and/or an electromagnetic signal (such as radio or light) receiver and/or transmitter which is indicated schematically at 202 (FIG. 3) which allows the system to work wirelessly. A wireless arrangement makes the entire BIA process a lot easier and simpler by eliminating the need for using wires between the instrument and the animal and thereby, also, reducing the dangers and problems associated with using wires around the animal.

For a further understanding of the nature, function, and objects of the present invention, reference should now be made to the following detailed description taken in conjunction with the accompanying drawings. Detailed descriptions of the preferred embodiments are provided herein, as well as, the best mode of carrying out and employing the present invention. It is to be understood, however, that the present invention may be embodied in various forms, Therefore, specific details disclosed herein are not to be interpreted as limiting, but rather as a basis for the claims and as a representative basis for teaching one skilled in the art to employ the present invention in virtually any appropriately detailed system, structure, method, process, or manner. The practice of the present invention is illustrated by the following examples, which are deemed illustrative of both the process taught by the present invention and of the results yielded in accordance with the present invention.

Functionally and in operation the present invention may be seen as a tetra-polar Bioelectrical Impedance Analysis system in which two surface electrodes at each end are employed to facilitate the impedance measurement. This system is intended for use primarily on hairy animals such as horses and the like.

The measurement of impedance is known and any of the many suitable systems may be used in the context of the present invention.

The electrodes need to be a set distance apart in order to provide reproducible measurements. In order to provide a good fit, vertical and horizontal independent movement is desired and provided.

Clips may be used to make good electrical contact with the electrode plates. Wireless transmission systems may be employed in connection with the present invention.

A variety of connecting rods, usually on the order of 5 to 10 cm, may be employed. Each rod needs to be suitably insulated and rods of various lengths provide for desired adjustment in length within the system.

Good electrical contact may be further facilitated by the use of known conducting creams and gels. Such creams and gels may be provided by use of a holder within or attached to the electrode structure.

Added flexibility may be obtained, if desired by use of ball and socket joints.

### Alternatives and Alternative Embodiments

While throughout this description, we have referred to various materials, chemicals, and apparatus as being presently preferred, it will be clear to one skilled in the art that other materials, chemicals, apparatus, methods, processes, steps and embodiments may be employed which will also provide the advantages as herein set forth in connection with the present invention. The present invention is not limited to the representative examples disclosed herein. Moreover, the scope of the present invention covers conventionally known variations and modifications to the system and the components described herein, as would be known by those skilled in the art. Such variations and equivalents are intended to be within the scope of the present invention. Accordingly, the invention is to be broadly construed and is to be limited only by the scope and spirit of the claims appended hereto.

To provide a description of the present invention that is both concise and clear, various examples of ranges have been set forth herein and in all cases should be read as though expressly identified with the phrase “including all intermediate ranges and combinations thereof”. Examples of specific values (e.g., ohms, ° C., μm, kg/L, volts, amps, current, intensity, etc.) that can be within a cited range by the reference to “including all intermediate ranges and combinations thereof” include 0.000001, 0.00001, 0.0001, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.1, 1.2, 1.30, 1.31, 1.32, 1.33, 1.34, 1.35, 1.36, 1.37, 1.38, 1.39, 1.40, 1.41, 1.42, 1.43, 1.44, 1.45, 1.46, 1.47, 1.48, 1.49, 1.50, 1.51, 1.52, 1.53, 1.54, 1.55, 1.56, 1.57, 1.58, 1.59, 1.60, 1.61, 1.62, 1.63, 1.64, 1.65, 1.66, 1.67, 1.68, 1.69, 1.70, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1050, 1100, 1150, 1200, 1250, 1300, 1350, 1400, 1450, 1500, 1600, 1700, 1800, 1900, 2000, 3000, 4000, 5000, 6000, 7000, 8000, 9000, 10,000, or more and so forth.

The above conventions may be understood by means of a number line a common element of elementary mathematics. It can be constructed by marking off two points: zero (the origin) and one (1). The distance from 0 to the point 1 is called the unit segment. The distance between all consecutive whole numbers is the same. When measurements fall somewhere between whole numbers. we may describe the situation in terms of a fractional length or in decimal terms of tenths, hundredths, thousandths and so forth. For example, if a measurement falls between 4 and 5, we may find that it is closer to 4.3 than 4.4. If we want more precision (and it is appropriate), we may continue to “zoom in” in which case we move more decimal places to the right. For numbers less than 5 in the relevant place one may round down and for numbers greater than 5 one may round up. When the relevant place contains a 5, the rule is to round so that the last nonzero digit is an even number. Whenever a range is given herein the above rules are intended to apply and the range is intended to cover all points on the number line from the lowest number to be rounded to the bottom of the range to the highest to the highest number to be rounded to the top of the range.

General ranges and the usual definitions for significant figures for each type of unit (e.g., ohms, %, ° C., μm, kg/L), are contemplated. Examples of values that can be within a cited percentage range, as applicable, include 0.001% to 100%, including all intermediate ranges and combinations thereof. Examples of values that can be within a thickness range (e.g., coating and/or film thickness upon a surface), as applicable, in micrometers (“μm”), that can be within a cited range include of 1 μm to 2000 μm, including all intermediate ranges and combinations thereof. Similar examples may be understood to apply to all of the units and systems of units mentioned above, such as ohms and the like or otherwise discussed below.

The following comments are intended to apply to all units and their conversions to whatever system of units including but not limited to length (m), mass (kg), time(s), speed, force, work, energy, heat, pressure including but not limited to angular frequency or velocity (radian/second), reactance (ohm), resistance (ohm) capacitance (farads), charge (coulomb), current (ampere), electromotive force (volt), work or energy (joule), force (Newton), frequency (Hertz), inductance (Henry), magnetic field (B, Tesla), Magnetic flux (Weber), potential (volt) power (waft), etc.

Specific units from one or more of the following systems may be used including but not limited to S.I., m.k.s. practical units; Gaussian units; Heaviside-Lorentz units; electrostatic units, and/or electromagnetic units.

In addition to the standard units the micron (μ=10⁻⁶ m) and Angstrom (Å=10⁻¹⁰ m) are frequently used and may be used herein.

The electrode may be hardwired to the appropriate electrical input for BIA or other measurements. Additionally the electrode holder can provide a base on which can be mounted an electro-magnetic energy power supply (such as electricity) and/or an electro-magnetic signal (such as radio or light) receiver and/or transmitter which allows the system to work wirelessly. A wireless arrangement makes the entire BIA process a lot easier and simpler by eliminating the need for using wires between the instrument and the animal and thereby, also, reducing the dangers and problems associated with using wires around the animal.

### SUMMARY

An electrode holder comprising a pair of electrodes connected by a flexible, insulating rod which controls the position of the electrodes with respect to each other.

It is noted that the embodiment described herein in detail for exemplary purposes is, of course, subject to many different variations in structure, design, application, and methodology. Because many varying and different embodiments may be made within the scope of the inventive concepts herein taught, and because many modifications may be made in the embodiment herein detailed in accordance with the descriptive requirement of the law, it is to be understood that the details herein are to be interpreted as illustrative and not in a limiting sense. It will be understood in view of the instant disclosure, that numerous variations of the invention are now enabled to those skilled in the art. Many of the variations reside within the scope of the present teachings. It is not intended to limit the scope of the invention to the particular forms set forth, but on the contrary, it is intended to cover such alternatives, modifications, and equivalents as may be included within the spirit and scope of the teachings and claims of the present invention. Accordingly, the invention is to be broadly construed and is to be limited only by the spirit and scope of the claims appended hereto.

What is claimed is:

1. An electrode holder comprising means for conducting current to a pair of electrodes, said pair of electrodes connected by a flexible, insulating rod which controls the position of the electrodes with respect to each other.

2. The electrode holder of claim 1 wherein two or more electrodes are connected by a flexible, insulating rod.

3. The electrode holder of claim 1 wherein the electrode is formed to conform to the topology of the animal in the section of the body where the electrodes are to be placed to make the desired electrical measurement, said electrode holder allowing for electrode movement to conform to the surface topology of the animal.

4. The electrode holder of claim 1 wherein the electrodes are held in position by a flexible rod and are limited in some degree of motion by that rod at a predetermined fixed distance between the electrodes.

5. The electrode holder of claim 1 wherein the electrodes have means to facilitate their being secured to the animal on whom the measurements are to be made without penetrating the skin of said animal.

6. The electrode holder of claim 1 wherein the electrode holder has means for transmitting and receiving signals from an electrical signal producer, measuring system, or Bioelectrical Impedance Analyzer.

7. The electrode holder of claim 1 wherein the electrode holder has means for receiving signals or power from an electrical signal producer, measuring system or Bioelectrical Impedance Analyzer via hard wires, or an electromagnetic power supply, or an electromagnetic signal receiver or transmitter or any combination of the above.

8. A method of Bioelectrical Impedance Analysis comprising:
(a) providing a voltage difference across two or more electrodes;
(b) maintaining said electrodes in a fixed spatial relation and in good electrical contact with the skin of the animal without penetrating the skin of the animal on which said measurements are to be made; and
(c) measuring the impedance of said animal in the region of or between the electrodes.

9. The method of claim 8 wherein the electrodes are spatially fixed by an electrode holder comprising a flexible, insulating rod connecting two or more electrodes.

10. The method of claim 8 wherein the electrodes conform to the topology of the animal in the region to be measured, by virtue of their size, shape and connection to the flexible rod of the electrode holder.

11. The method of claim 8 where the electrodes are coated with a layer of cream or gel to increase electrical contact with the skin.

12. The method of claim 8 wherein the electrode holder has means for receiving signals or power from an electrical signal producer, measuring system or Bioelectrical Impedance Analyzer via hard wires, or an electromagnetic power supply, or an electro-magnetic signal receiver or transmitter, or any combination of the above.
